(19) United States
(12) Patent Application Publication
(10) Pub. No.: US 2022/0160488 A1
(43) Pub. Date: May 26, 2022

(54) FUNCTIONALIZED PROSTHETIC INTERFACES FOR THE PREVENTION AND TREATMENT OF DENTAL CONDITIONS

(71) Applicant: OPTIMED TECHNOLOGY, INC., North Tonawanda, NY (US)

(72) Inventors: Praveen ARANY, Amherst, NY (US); Malvika NAGRATH, Brampton (CA); Saeed Ur RAHMAN, Swabi, KP (PK); Jacob GRACA, Wilson, NY (US); Alexander J. SIKORA, Lake View, NY (US); Sharaschandra Reddy GOVINDOOL, Bangalore (IN)

(21) Appl. No.: 17/437,187

(22) PCT Filed: Mar. 9, 2020
(86) PCT No.: PCT/US2020/021772

§ 371 Date: Sep. 8, 2021

Related U.S. Application Data
(60) Provisional application No. 62/815,671, filed on Mar. 8, 2019.

(51) Int. Cl. A61C 19/06; A61C 5/00; A61C 13/01; A61C 13/00; A61C 9/00; A61K 31/7048; A61L 27/26; B33Y 80/00; B33Y 10/00

(52) U.S. Cl. CPC A61C 19/063; A61C 5/007; A61C 13/01; A61C 13/0019; A61C 9/0006; A61L 2430/12; A61K 31/7048; A61L 27/26; B33Y 80/00; B33Y 10/00; A61C 9/0053

(57) ABSTRACT

Provided are compositions and devices containing microspheres encapsulating one or more drug(s). The compositions and devices may be used to treat or prevent infection associated or caused by oral mucosal diseases, oral infections, oral conditions, dental conditions, or surgical recovery. The devices include, but are not limited to, splints, mouth guards, full dentures, partial dentures, or fillings. The devices may be manufactured from filaments that have a composition of the present disclosure. Also provided are methods of making and using the devices and compositions.

### CROSS REFERENCE TO RELATED APPLICATIONS

This application claims priority to U.S. Provisional Application No. 62/815,671, filed on Mar. 8, 2019, the disclosure of which is incorporated herein.

### BACKGROUND OF THE DISCLOSURE

The global biomaterial consumables market was worth about US $66.2 billion in 2015 and is expected to grow about 14% by 2020. A large part of this industry is focused on the use of dental polymers and a primary focus has been on the fabrication of dentures. It was predicted that the total population of edentulous adults in the US is going to be 8.6 million by 2050. The American College of Prosthodontists state that about 90% of edentulous patients utilize dentures as rehabilitation treatment. Besides innovations in assessments and fabrications, dentures made of poly-methyl methacrylate (PMMA) are the mainstay in prosthetic dentistry. However, a major concern among these denture wearers, especially aged folks with limited manual dexterity to clean prosthesis, is the presence of Candida albicans (C. albicans) leading to denture stomatitis with a reported prevalence of over 65% among denture-wearing population. Candidal infection progresses through three stages namely, palatal petechiae, diffuse erythema, and papillary hyperplasia mostly in the central areas of hard palate and alveolar ridge. This disease is of multifactorial origin with numerous local and systemic factors. Current treatment options include disinfecting agents, antiseptic mouthwashes, antifungal agents, microwave disinfection, and photodynamic therapy.

Despite these treatment options, the multifactorial nature and complexity of biofilm-tissue interactions has limited clinical efficacy of denture-associated oral candida infections. Further, differences in the surface charge between PMMA (negative) and denture pellicle (positive) predisposes them to colonization with biofilms including C. albicans. Various techniques that modify PMMA surface charge, such as carboxylate and phosphate anions or co-polymerization with anionic monomers, have been attempted but have been noted to modify physical properties of PMMA. Polycaprolactone (PCL) is another biocompatible and biodegradable polymer with low permeability. It has been used as micro or nanospheres for sustained delivery of biomolecules such as peptides or drugs.

Advances in digital technologies such as Computer Aided Designing and Computer Aided Machining (CAD-CAM) are being widely adapted in clinical dentistry. A few advantages these digital approaches offer compared to conventional denture processing include elimination of polymerization shrinkage of PMMA dentures ensuring better fit, easier duplication of the dentures for better clinical adaptation of new dentures, and reduced surface porosity that results in lower microbial retention on its surface. Also, fabrication of dentures with a consistent minimal thickness of denture bases, reduced clinical appointments, treatment time, and cost reduction without compromising quality, improved communication and satisfaction of these approaches by patients and clinicians are seen. Besides these advantages, digital workflow for prostheses integrates effectively with development of electronic health records.

3D printing approaches are broadly divided into additive and subtractive techniques. Clinical dentistry was an early adapter of the subtractive, including milling technique and digital imaging for tooth crown fabrication. Additive techniques have, unfortunately, lagged behind despite significant progress in technology due to a lack of guidelines and regulation of dental materials. Hence, innovations in biomaterials offering additional functionality will spur further acceptance and popularity of dental 3D printing.

Therefore, what is needed is an improved interface for dentures and other prosthetics for the delivery of drugs or other compounds for the prevention and treatment of oral and dental conditions.

### BRIEF SUMMARY OF THE DISCLOSURE

3D printing using a fused filament fabrication printer of a PMMA polymer filament are disclosed. The filament contains an antimicrobial agent (e.g., Amphotericin-B) encapsulated in polycaprolactone (PCL) microspheres. These microspheres are biocompatible, biodegradable, and a suitable candidate for sustained and controlled drug release in the vicinity of the infection (generally mucosa below the denture). The 3D printed prosthesis is fabricated in layers, providing the intaglio and occlusal surface of the denture to be antimicrobial, while the body of the denture is made of PMMA without the drug. This can be used for fabrication of microbial infection-resistant surgical splints; complete or removable dentures; or other devices or prostheses for immunocompromised (e.g., an HIV-positive individual), hospitalized, elderly, or disabled patients. This drug containing PCL microsphere layer also can be printed onto the surface of previously existing dentures of individuals with other oral or dental conditions.

Fabrication of 3D printed dentures from custom-fabricated PMMA polymer filaments using a syringe extrusion technique is disclosed. Further, PCL microspheres that encapsulated an antimicrobial agent were synthesized and layered onto PMMA polymer 3D printing denture surfaces that contact tissues. Using a fused filament thermoplastic process, dentures were 3D printed to replicate accurate anatomy and tested for mechanical properties compared to conventional dentures. Studies to characterize sustained antimicrobial drug release from microspheres and denture surface were performed. Finally, 3D printed surfaces were colonized with C. Albicans and reduction of 3D-printed functionalized surfaces was noted in a biomass assay. This study demonstrates the feasibility of 3D printing dental prostheses with functionalized surfaces that can actively neutralize oral infections or alleviate other oral and dental conditions.

In an aspect this disclosure provides a method for synthesizing 3D printed polymer dental prostheses containing microspheres loaded with compounds for alleviating medical conditions associated with the oral cavity and/or dental tissues.

In an aspect this disclosure provides a method for rebuilding previously existing dental prostheses using 3D printed polymers containing microspheres loaded with compounds to alleviate medical conditions associated with the oral cavity and/or dental tissues.

In an aspect this disclosure provides a method for treating an individual afflicted with a microbial infection of the oral cavity comprising the following: i) loading microspheres with antimicrobial compounds; ii) synthesizing a 3D printed polymer dental prosthesis or relining a previously existing dental prosthesis with a 3D printed polymer containing antimicrobial loaded microspheres; and iii) administering to an individual the 3D printed dental prosthesis containing the antimicrobial compound loaded microspheres.

### DESCRIPTION OF THE DRAWINGS

For a fuller understanding of the nature and objects of the disclosure, reference should be made to the following detailed description taken in conjunction with the accompanying drawings.

FIG. 1 illustrates mechanical testing: flexural strength of conventionally processed and 3D printed PMMA prepared as per ISO4049 standards. n=8, *p<0.05, T test.

FIG. 2 illustrates PMMA functionalization for a 3D printed prosthesis loaded with fluorescent microspheres containing Protoporphyrin X (PpX). Upper panels show a digital image of the 3-unit partial denture and lower image shows fluorescence images. Broken outline of prostheses are noted.

FIG. 3 illustrates drug release: Polycaprolactone (PCL) microsphere encapsulated Amphotericin B (AmB) drug release was characterized by Fourier Transform Infrared Spectroscopy (FTIR) and spectrophotometry. a. FTIR analyses were performed on PMMA discs where a. AmB drug, b. AmB loaded PMMA and c. unloaded PMMA. b. Spectrophotometry was performed on 3D printed 1, 5 or 10 layered discs that were incubated in release media over 1, 3 and 5 days. Percent transmission was assessed at 344-355 nm region where AmB alone demonstrated discrete peaks. c. Electron microscopic images of PCL microspheres immediately after fabrication (left panel) and following long term storage leading to surface degradation (right panel)leading to drug release.

FIG. 4 illustrates Candida albicans biomass assay: The antifungal properties of 3D printed PCL-PMMA was examined in a biomass assay a. To assess antifungal effects, 3D printed 1, 5 and 10 layered sample were incubated with C. albicans for 48 h and dry weight of their biofilm biomass was assessed. n=3, *p<0.05, T test. b. Electron microscopy of 1 (left), 5 (middle) and 10 (right) layered AmB containing PCL microspheres incorporated PMMA disc. c. AmB was heat-treated at various temperatures and placed on candida cultures and examined after 24 h for efficacy (left panel). Digital images were captured and the cleared zone was quantitated (right panel) n=2, *p<0.05, T test.

FIG. 5 illustrates the generation of PMMA filaments with PCL-laden microspheres, which allows incorporation of biological payloads enabling 3D printing of functionalized prosthesis surfaces.

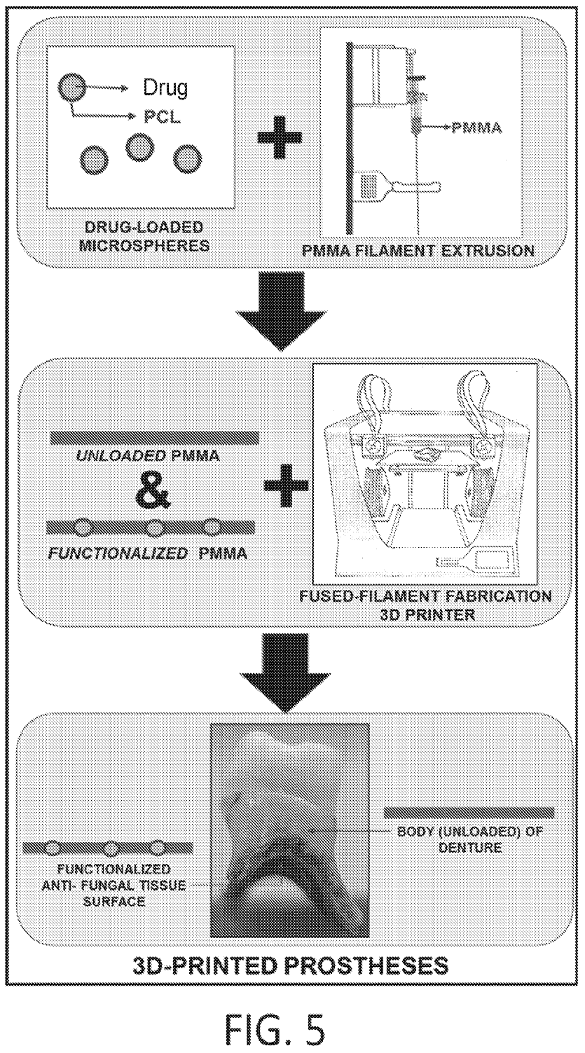

FIG. 6 is an SEM image of the BSA incorporated PCL microspheres.

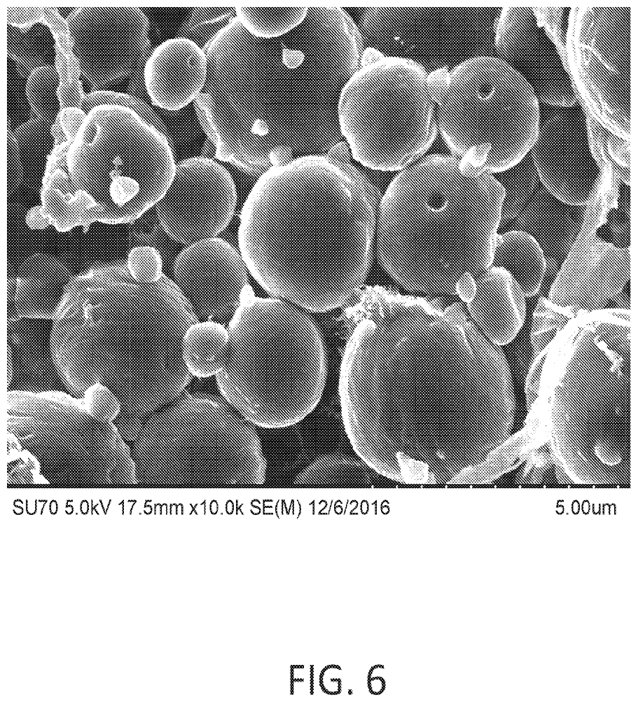

FIG. 7 illustrates extrusion of the filament.

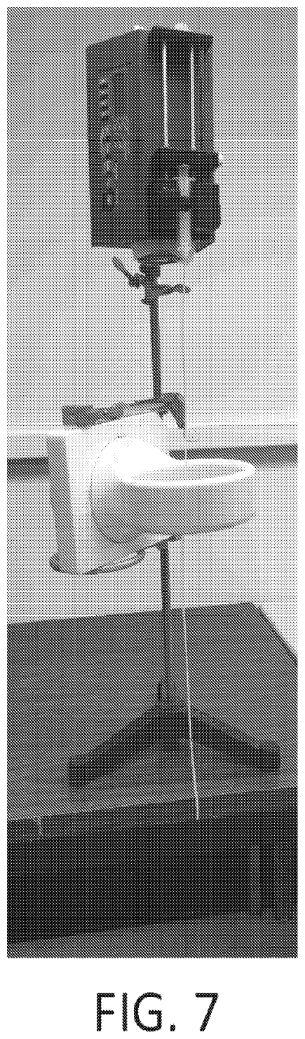

FIG. 8 illustrates a BCN3D SIGMA fused filament fabrication printer.

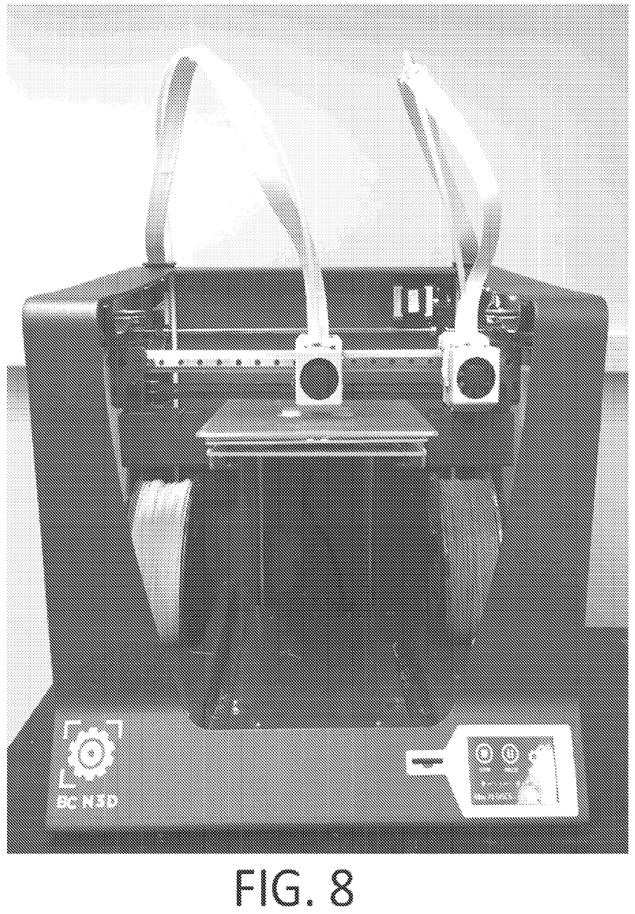

FIG. 9 illustrates a fluorescent image of PCL microspheres containing Protoporphyrin-IX-fluorescent dye.

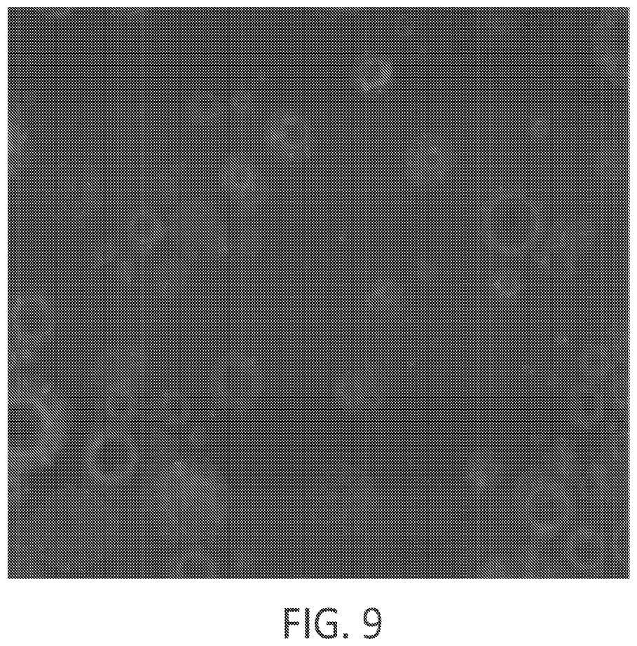

FIG. 10 illustrates the 3D printed 3-unit denture 1 (PMMA) and 2 (PMMA with PCL microspheres containing fluorescent agent-Protoporphyrin-IX) under different wavelength exposure of 800 nm (A) and 480 nm (B) confirms the presence of the microspheres in the PMMA filament. In (A), which is in Chemidoc Bio RAD system, both control and experimental sample were exposed to 800 nm light. The resultant image shows radiance by the PMMA material. In (B), which is in Chemidoc Bio RAD system both control and experimental samples were exposed to 480 nm light to cause fluorescence of dye contained within PCL microspheres. No fluorescence was exhibited in the control and significant fluorescence was exhibited in the experimental.

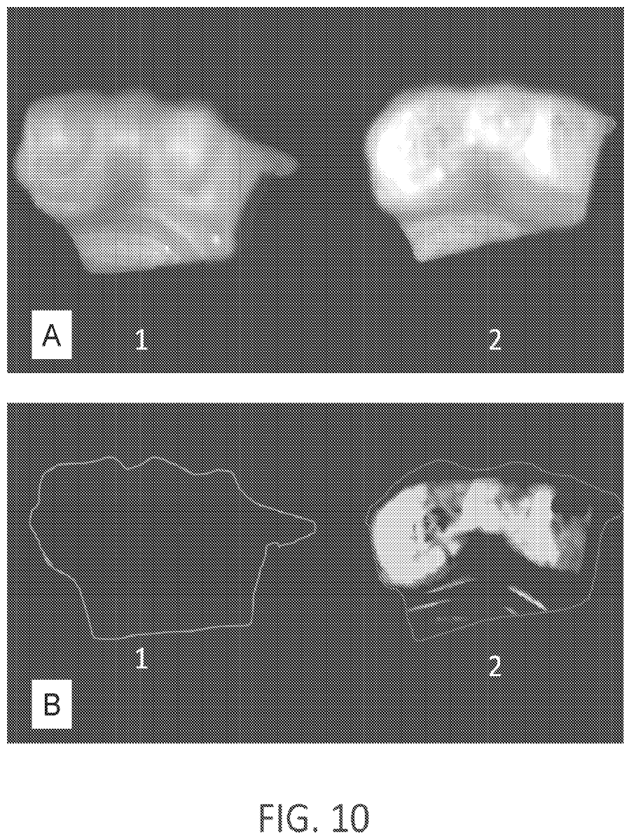

FIG. 11 is a BSA release study. The graph depicts the release of the protein from the PCL microspheres in the PMMA discs over a period of 21 days. The release of the drug is released increasingly over the period of the time.

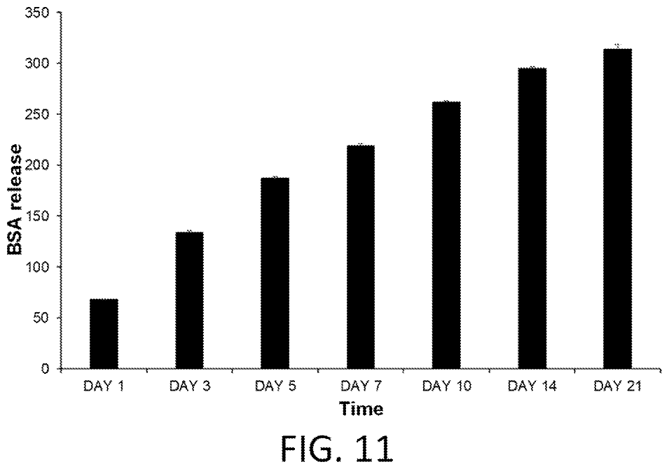

FIG. 12 is a table showing the biofilm biomass. Control discs showed reduction in the mass after the experiment indicating towards their degradation in the media. The 10-layered experimental discs showed no biofilm biomass formed on the surface indicating towards their resistance to biofilm formation.

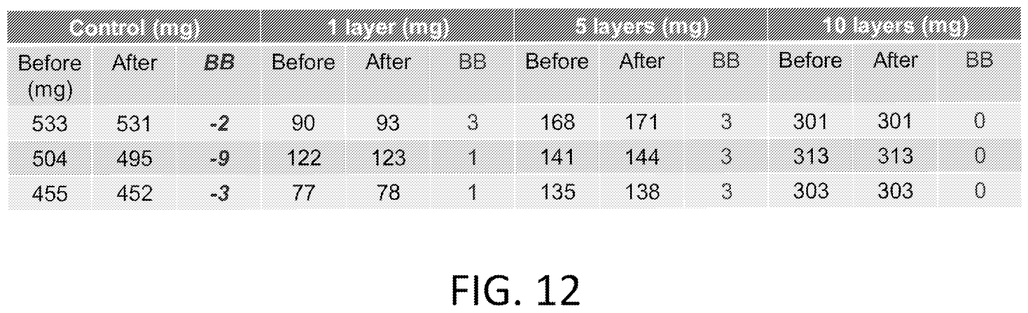

### DETAILED DESCRIPTION OF THE DISCLOSURE

Although claimed subject matter will be described in terms of certain embodiments, other embodiments, including embodiments that do not provide all of the benefits and features set forth herein, are also within the scope of this disclosure. Various structural, logical, and process step changes may be made without departing from the scope of the disclosure.

Disclosed herein is a printer filament with active payloads. 3D printing can generate any conformation with micron level accuracy. Thus, embodiments of the dentures or other prostheses disclosed herein can deliver smart and/or active biomaterials for additional therapeutic benefits.

As used in this disclosure, the singular forms “a”, “an”, and “the” include plural references and vice versa, unless the context clearly dictates otherwise. Thus, for example, references to “the method” includes one or more methods, and/or steps of the type described herein.

The terms “oral cavity” and “oral” as used herein refers to all of the anatomical structures that make up the mouth and oropharynx of an individual such as but not limited to the lips, cheeks, tongue, hyoid bone, teeth, gums, jaw bone (mandible), alveolar bone, salivary glands, tonsils, adenoids, hard and soft palate, uvula, tempomandibular joint, epiglottis, and all connective and epithelial tissues.

The terms “infectious disease” and “microbial infection” as used herein refers to all diseases caused by infectious agents that include those of bacterial (e.g., Streptococcus sp. Staphylococcus sp, Haemophilis sp, Treponema pallidum (syphilis), Neisseria gonorrhoeae (oral gonorrhea), etc.), viral (e.g., herpes simplex virus, human papilloma virus, HIV, Coxsackie virus, etc.), fungal (e.g., Candida albicans, Histoplasma capsulatum, Blastomyces dermatitidis, Coccidiodies sp, and Aspergillus sp., etc.), or parasitic origin (e.g., Entameobagingivalis, Trichomonas tenax, and Leishmania sp, etc.).

The term “antimicrobial” and “antimicrobial agents” as used herein refers to drugs or methods used to treat medical conditions resulting from microbial infection. Anti-microbial agents can include antibiotics (e.g., penicillin, amoxicillin, azithromycin, clindamycin, cephalexin, trimethoprim, etc.), antivirals (e.g., acyclovir etc.), antifungals (e.g., amphotericin B, flucytosine, fluconazole, ketoconazole, miconazole, terbinafine, griseofulvin etc.) and antiparastitics (e.g., metronidazole, tinidazole, miltefosine, meglumine antimoniate, etc.)

The term “oral condition” or “dental condition” as used herein refers to any condition occurring in the oral cavity that can be caused by infectious microorganisms (e.g., dental caries, gingivitis, periodontal disease, hand foot and mouth disease, thrush, herpangina, canker sores, oral herpes, etc.) and/or surgical procedures (e.g., tonsillectomy, tooth extraction, root canal, tumor debulking, dental implantation, bone engraftment/repair, oral and maxillofacial surgery, etc.).

The term “Oral mucosal disease” or “Oral infections” as used herein refers to any disorders of the oral mucosa that can be causes by different reactions or infections (e.g., mucous membrane pemphigoid, oral lichen planus, chronic aphthous stomatitis, mucosal pemphigus vulgaris, and the like, and combinations thereof), autoimmune disease, or Graft versus host disease, or the like, or a combination thereof.

In an aspect, the present disclosure provides a composition comprising a one or more microsphere(s) comprising one or more drug(s) (e.g., antimicrobial agent(s), antifungal agent(s), antibiotic(s), antiviral(s), analgesic(s), steroid(s) (e.g., corticosteroid(s)), immune modulator(s), fluoride, and the like, and combinations thereof). The microspheres may encapsulate the one or more drug(s). The one or more encapsulated drug(s) may be the same or different. A filament may comprise the composition.

The microspheres may be formed from (e.g., comprise) various polymeric materials. Non-limited examples of polymeric materials include polyesters and the like. Examples of polyesters include, but are not limited to, polycaprolactone (PCL), polylactic acid, and the like, and combinations thereof. The microspheres of the present disclosure may be formed from chitosan, polyethylene, glass, polyphosphazene, or polymethacrylate, or be formed from liposomes. The microspheres are biocompatible and biodegradable. The microspheres may be suitable for sustained and controlled drug release in the vicinity of an infection.

While 3D printing is disclosed, the microspheres comprising an antimicrobial agent can be manually added to a prosthesis.

The drug may be various antimicrobial agents. Examples of the antimicrobial agent include antibiotics, such as, for example, penicillins (e.g., Flucloxacillin (Flopen, Flucil), Amoxicillin+clavulanate (Augmentin, Clamoxym), Piperacillin+tazabactam (Tazocin)), cephalosporins (e.g., Cephalexin (Keflex, Ibilex), Cephazolin (Kefzol), Ceftriaxone (Rocephin)), macrolides (e.g., Azithromycin (Zithromax), Roxithromycin (Rulide)), azoles (e.g., Fluconazole (Diflucan), Voriconazole (Vfend)), and guanine analogues (e.g., Aciclovir (Zovirax), Valaciclovir (Valtrex)). Additional antimicrobial agent examples include, but are not limited to, Aminoglycosides, Ansamycins, Carbacephem, Carbapenems, Cefepime, Ceftaroline, Ceftazidime, Ceftobiprole, Ceftobiprole, Ceftolozane/tazobactam, Clindamycin, Dalbavancin, Daptomycin, Doxycyline, Fluoroquinolones, Fusidic acid, Glycopeptides, Lincosamides, Linezolid, Lipopeptide, Monobactams, Mupirocin, Nitrofurans, Omadacycline, Oritavancin, Oxazolidinones, Piperacillin/tazobactam, Polypeptides, Quinolones/Fluoroquinolones, Streptogramins, Sulfonamides, Tedizolid, Telavancin, Tetracyclines, Ticarcillin/clavulanic acid, Tigecycline, Trimethoprim/sulfamethoxazole, Vancomycin, Gentamicin, Ampicillin, Amikacin, Aztreonam, Ciprofloxacin, Cefotaxime, Cefuroxime, Cefazolin, Imipenem, Levofloxacin, Meropenem, Tobramycin, Telithromycin, Doripenem, Retapamulin, Fidaxomicin, Curcumin, and the like, and combinations thereof.

The drug may be various growth factors. Non-limiting factors of growth factors include PDGF, EGF, TGF-α, TGF-β, KGF, FGF, IL-1, IGF-1, VEGF, BMPs, Wnts, IGF, IL-2, IL-3, IL-4, IL-5, IL-6, IL-7, LIF, IGF-2, MSP, MSF, TGF-α, Ang, AM, GDF-8, NGF, SHH, NGF, PDGF, FGFR, TNF, and the like, and combinations thereof.

The drug may be various antifungal agents. Non-limiting examples of antifungal agents include amphotericin B, flucytosine, fluconazole, ketoconazole, miconazole, terbinafine, griseofulvin, Clotrimazole, Econazole, Anidulafungin, Micafungin, Caspofungin, Butoconazole. Bifonazole, Clotrimazole, Econazole. Fenticonazole, Isoconazole, Ketoconazole, Luliconazole, Miconazole, Omoconazole, Oxiconazole, Sulconazole, Sertaconazole, Tioconazole, Albaconazole, Abafungin, amorolfin, butenafine, naftifine, terbinafine, Epoxiconazole, Efinaconazole, Fluconazole, Itraconazole, Isavuconazole, Propiconazole, Posaconazole, Ravuconazole, Terconazole, Voriconazole, and the like, and combinations thereof. The antifungal agents may be suitable to treat C. albicans.

The drug may be various analgesics. Non-limiting examples of analgesics include ibuprofen, acetaminophen, naproxen, lidocaine, benzocaine, salicylic acid, aspirin, Diflunisal, Diclofenac, Etodolac, Flurbiprofen, Fenoprofen, Indomethacin, Ketorolac, Meloxicam, Mefenamic acid, Naproxen, Oxaprozin, Nabumetone, Sulindac, Piroxicam, Tolmetin, and the like, and combinations thereof.

The drug may be various steroids. In various examples, the steroids are corticosteroids. Non-limiting examples of corticosteroids include prednisone, prednisolone, dexamethasone, methylprednisone, bethamethasone, and the like, and combinations thereof. The steroids may include, but are not limited to, Anabolic Androgenic Steroids, Glucocorticosteroids, or Minerocorticosteroids. Various other suitable steroids include, but are not limited to, Testosterone (e.g., Axiron, Androgel, Fortesta, Testopel, Striant, Delatestryl, Testim, Androderm), Androstenedione, Stanozolol (Winstrol), Nandrolone (Deca-Durabolin), Methandrosteolone (Dianabol), triamcinolone, fluocinolone, betamethasone, halcinonide, fluocinonide, hydrocortisone, diflorasone, clobetasol, desoximetasone, desonide, prednicarbate, fluticasone, mometasone, flurandrenolide, halobetasol, alclometasone, amcinonide, and the like, and combinations thereof.

The drug may be various immune modulators. Non-limiting examples include Bacillus Calmette Guerin (BCG), Levamisole, Recombinant Cytokines (e.g., Interferons, Interleukins, Colony stimulating factors), Thalidomide Isoprinosine (e.g., Immunocynin), other drugs (e.g., Inosiplex, Azimexon, Imexon, Thymosin, Methylinosine, Monophosphate), immunization (e.g., Vaccines, Immune Globulin), Anakinra, Etanercept, adalimumab, alefacept, abatacept, efalizumab, rituximab, natalizumab, ustekinumab, golimumab, Methotrexate, Azathioprine, Cylcosporine, tocilizumab, and the like, and combinations thereof.

The drug may be fluoride. Without intending to be bound by any particular theory, it is considered fluoride-loaded microspheres are suitable for preventing plaque development and tooth decay.

In an aspect, the present disclosure provides methods for synthesizing 3D-printed polymer dental prostheses, which may comprise one or more microsphere(s) comprising one or more drug(s). The dental prostheses may be suitable to rebuild previously existing dental prostheses or for new prostheses.

In an embodiment, the method is used to synthesize 3D printed polymer surgical splints containing microsphere encapsulated growth factors (e.g., PDGF, EGF, TGF-α, TGF-β, KGF, FGF, IL-1, IGF-1, VEGF, and the like (such as, for example, those described herein), and combinations thereof) for the purpose of enhancing wound healing in the oral cavity as a result of an oral or dental condition such as but not limited to surgery or infection.

In an embodiment, the method is used to synthesize 3D printed polymer orthodontic appliances containing fluoride loaded microspheres for the purpose of preventing plaque development and tooth decay.

In an embodiment, the method is used to synthesize 3D printed polymer interim treatment prostheses containing microspheres loaded with analgesics (e.g., ibuprofen, acetaminophen, naproxen, lidocaine, benzocaine, salicylic acid, aspirin) for the purpose of relieving pain and inflammation associated with an oral or dental condition, such as but not limited to surgery or infection.

In an embodiment, the method is use to synthesize interim treatment prostheses containing microspheres loaded with corticosteroids (e.g., prednisone, prednisolone, dexamethasone, methylprednisone, or bethamethasone) for the purpose of relieving pain and inflammation associated with an oral or dental condition such as but not limited to surgery or infection. The steroids contained in the microspheres can include Anabolic Androgenic Steroids, Glucocorticosteroids, or Minerocorticosteroids. The microspheres also can include Testosterone (e.g., Axiron, Androgel, Fortesta, Testopel, Striant, Delatestryl, Testim, Androderm), Androstenedione, Stanozolol (Winstrol), Nandrolone (Deca-Durabolin), or Methandrosteolone (Dianabol).

In an embodiment, the prostheses contain microspheres loaded with immune modulators such as Bacillus Calmette Guerin (BCG), Levamisole, Recombinant Cytokines (e.g., Interferons, Interleukins, Colony stimulating factors), Thalidomide Isoprinosine (e.g., Immunocynin), other drugs (e.g., Inosiplex, Azimexon, Imexon, Thymosin, Methylinosine, Monophosphate), or immunization (e.g., Vaccines, Immune Globulin).

In an embodiment, the method is used to treat an individual recovering from a surgery or dental procedure in the oral cavity. The individual will be administered a personalized 3D printed PMMA polymer dental prosthesis coated in PCL microspheres that contain a suitable drug (e.g., analgesics, corticosteroids, growth factors, and the like, and combinations thereof) either alone or in combination with other drugs (e.g., antibiotic, antifungal, antiviral, antiparasitic, and the like) to treat a specific infection or oral or dental condition.

In an embodiment, the method is used to treat an individual afflicted with an infection of the oral cavity. The individual will be administered a personalized 3D printed PMMA polymer dental prosthesis coated in PCL microspheres that contain a suitable antimicrobial agent (e.g., antibiotic, antifungal, antiviral, antiparasitic, and the like, and combinations thereof) either alone or in combination with other antimicrobial agents or other drugs (e.g., analgesics, corticosteroids, growth factors, and the like, and combinations thereof) to treat a specific infection or oral or dental condition, such as those described herein.

In an embodiment, the infectious disease-causing agent in the oral cavity is Candida albicans. C. albicans is normally a commensal colonizer of the oral cavity; however, outgrowth of the fungus causes a condition known as thrush. Causes of thrush include antibiotic treatments; a compromised or under developed immune system (e.g., HIV-positive individual); and chemotherapy and radiation treatments for cancer. Thrush is characterized by the development of patchy, white, “curd-like” lesions on the tongue, cheeks, palate, and back of the mouth. Individuals who wear dentures or dental prostheses are at increased risk for the development of thrush.

In an embodiment, the treatment of the oral or dental condition is microspheres containing encapsulated anti-fungal agents (e.g., amphotericin B, flucytosine, fluconazole, ketoconazole, miconazole, terbinafine, griseofulvin, and the like (such as, for example, those described herein), and combinations thereof) designed to treat C. albicans infections.

Various prostheses may be produced by a method of the present disclosure. Non-limiting examples of prostheses (e.g., dental devices or simply devices) include microbial infection-resistant surgical splints; fillings; mouth guards; complete or removable dentures; or other devices or prostheses. The dentures may be full or partial dentures.

In various examples, only the surface of the dentures includes the microspheres or other antimicrobial agent.

In an embodiment, these dental prostheses or dentures can be provided as “treatment dentures” for a short duration of timing for the treatment of an infectious disease; assisted wound healing resulting from infection, injury, or surgery; and/or for the prevention of plaque development and tooth decay.

In an embodiment, these dental prostheses or dentures can be provided as “preventative dentures” or “prophylactic dentures” for a long duration of timing for long-term disease prevention or treatment; assisted wound healing resulting from infection, injury, or surgery; and for the prevention of plaque development and tooth decay.

Embodiments disclosed herein can be used to provide the inner layers of the printed cast for broken bone stabilization and alignment with medication for pain relief or in case of compound fractures deliver medication for skin or bone healing.

Embodiments disclosed herein also can be used to provide patches or bandages for contact dermatitis or mucositis using appropriate medication. The patches or bandages may be provided in the form of microspheres-incorporated nanofibrous matrices for contact dermatitis or mucositis. The embodiments can be also used to provide the drug-loaded buccal films for the prevention or treatment of wound healing from infection or injury. Examples of infections are described herein.

The antimicrobial agent can be provided 2 folds+ED50 concentration, but other concentrations are possible.

In an instance, light-activated biomolecules (e.g., protoporphyrins, porphyrins, and other photodynamic therapies) can be incorporated in the microspheres and can be activated for the local delivery from the prosthesis. Irradiation may induce release of the one or more drug(s) and/or induce biological activity of the one or more drug(s). Examples of light-activated biomolecules and complexes include, but are not limited to, fluorescent conjugated proteins, methylene blue, pheophorbides, anthraquinones, xanthenes, curcuminoids, ruthenium complexes, iridium complexes, and the like, and combinations thereof. Light-activated biomolecules and complexes may be incorporated into the microspheres and/or activated for the local delivery. Activation may occur for the local delivery of different diseases.

Capture and sense systems also can be incorporated within 3D printed dental prostheses for diagnoses and treatments of oral or dental conditions. For example, this method can be used for detection and treatment of a fungal infection by using a 3D printed polymer dental prosthesis containing antibody tagged nanowall carbon tubes coupled with a release mechanism of antifungal agents.

In an embodiment the method to create the 3D printed dental prosthesis comprises the following:

(a) Obtaining a scan, mold, or model of the individual's oral cavity where the dental prosthesis will fit;

(b) Using a 3D printer containing PMMA polymer, print a core dental prosthesis corresponding to the individual's scan, mold, or model of the oral cavity;

(c) Generating microspheres comprised of PCL that contain a compound or compounds (e.g., Amphotericin-B) to treat or prevent a dental condition; and

(d) Coating the 3D printed PMMA core dental prosthesis with compound loaded PCL microspheres.

In an embodiment, the method to create the 3D printed dental prosthesis comprises:

(a) Obtaining a scan, mold, or model of the individual's oral cavity where the dental prosthesis will fit;

(b) Using a 3D printer containing a PMMA polymer filament to print a core dental prosthesis corresponding to the individual's scan, mold, or model of the oral cavity, where the PMMA polymer filament comprises one or more microsphere(s) comprising one or more drug(s) (e.g., antimicrobial agent(s), antifungal agent(s), antibiotic(s), antiviral(s), analgesic(s), steroid(s) (e.g., corticosteroid(s)), immune modulator(s), fluoride, and the like, and combinations thereof). The microspheres may encapsulate the one or more drug(s). The one or more encapsulated drug(s) may be the same or different.

In an embodiment, the method to treat an individual afflicted with an oral or dental condition using a 3D printed dental prosthesis comprises the following:

(a) Obtaining a scan, mold, or model of the individual's oral cavity where the dental prosthesis will fit;

(b) Using a 3D printer containing PMMA polymer, print a core dental prosthesis corresponding to the individual's scan, mold, or model of the oral cavity;

(c) Generating microspheres comprised of PCL, containing the compound or compounds necessary to treat or prevent the individual's oral or dental condition, such as, Amphotericin-B loaded microspheres to treat an oral C. albicans infection;

(d) Coating the 3D printed PMMA core dental prosthesis with compound loaded PCL microspheres;

(e) Administering the completed dental prosthesis to the afflicted individual; and

(f) Monitoring the individual's response to the dental prosthesis treatment.

In an embodiment, the method to treat an individual afflicted with an oral or dental condition using a 3D printed dental prosthesis comprises the following:

(a) Obtaining a scan, mold, or model of the individual's oral cavity where the dental prosthesis will fit;

(b) Using a 3D printer containing a PMMA polymer filament to print a core dental prosthesis corresponding to the individual's scan, mold, or model of the oral cavity, where the PMMA polymer filament comprises one or more microsphere(s) comprising one or more drug(s) (e.g., antimicrobial agent(s), antifungal agent(s), antibiotic(s), antiviral(s), analgesic(s), steroid(s) (e.g., corticosteroid(s)), immune modulator(s), fluoride, and the like, and combinations thereof). The microspheres may encapsulate the one or more drug(s). The one or more encapsulated drug(s) may be the same or different, the microspheres may be, for example, Amphotericin-B loaded microspheres to treat an oral C. albicans infection;

(c) Administering the completed dental prosthesis to the afflicted individual; and

(d) Monitoring the individual's response to the dental prosthesis treatment.

In various examples, the individual treated in a method of the present disclosure has (e.g., is diagnosed with) or is suspected of having an oral condition, dental condition, an oral mucosal disease, oral infection, or is recovering from surgery. Non-limiting examples of oral conditions and/or dental conditions include conditions caused by and/or associated with infectious microorganisms. Non-limiting examples of conditions include dental caries, gingivitis, periodontal disease, hand foot and mouth disease, thrush, herpangina, canker sores, oral herpes, and the like, and combinations thereof. Non-limiting examples of oral mucosal diseases and/or oral infections include disorders of the oral mucosa caused by different reactions or infections, auto-immune diseases, or Graft versus host disease. Non-limiting examples of reactions or infections include mucous membrane pemphigoid, oral lichen planus, chronic aphthous stomatitis, and mucosal pemphigus vulgaris. Non-limiting examples of surgical procedures that an individual may be recovering from include tonsillectomy, tooth extraction, root canal, tumor debulking, dental implantation, bone engraftment/repair, oral and maxillofacial surgery, and the like, and combinations thereof.

This disclosure provides a method for the construction of a 3D printed polymer dental prosthesis coated with PCL microspheres containing compounds; compositions comprising any of the foregoing; methods of making any of the foregoing; and methods of using any of the foregoing to treat a condition of the oral cavity.

The following Statements provide examples of the present disclosure.
Statement 1. A device comprising the following: (a) a prosthesis; and (b) polycaprolactone (PCL) microspheres loaded with a compound disposed in the prosthesis to treat oral or dental conditions.
Statement 2. The device according to Statement 1, where the 3D printed prosthesis is a denture.
Statement 3. The device according to Statement 1 or Statement 2, where the compound in PCL microspheres is an antimicrobial agent.
Statement 4. The device according to any one of the preceding Statements, where the antimicrobial agent is the antifungal Amphotericin B.
Statement 5. The device according to any one of the preceding Statements, where the 3D printed prosthesis includes polymethylmethacrylate (PMMA) polymer filaments.
Statement 6. The device according to any one of the preceding Statements, where the compound is a drug used to recover from a surgery or dental procedure.
Statement 7. The device according to Statement 7, where the compound is an analgesic, corticosteroid, or growth factor designed to alleviate pain and inflammation and enhance wound healing.
Statement 8. The device according to any one of the preceding Statements, where the prosthesis is 3D printed.
Statement 9. A method of making a device comprising: (a) obtaining a scan, mold, or model of the individual's oral cavity where the dental prosthesis will fit; (b) fabricating a core dental prosthesis corresponding to the individual's scan, mold, or model of the oral cavity with PMMA polymer; (c) generating microspheres comprised of PCL that contain a compound or compounds to treat or prevent an oral or dental condition; and (d) coating the PMMA core dental prosthesis with compound loaded PCL microspheres.
Statement 10. The method according to Statement 9, where the prosthesis is a denture.
Statement 11. The method according to Statement 9 or Statement 10, where the compound is an antimicrobial agent.
Statement 12. The method according to Statement 11, where the antimicrobial agent is the antifungal agent Amphotericin B.
Statement 13. The method according to any one of Statements 9-11, where the compound is a drug used to recover from a surgery or dental procedure.
Statement 14. The method according to any one of Statements 9, 10, or 13, where the compound is an analgesic, corticosteroid, or growth factor designed to alleviate pain and inflammation and enhance wound healing.
Statement 15. The method according to any one of Statements 9-14, where the fabricating includes 3D printing.
Statement 16. A method of treating an individual afflicted with a dental condition using a dental prosthesis comprising the following: (a) obtaining a scan, mold, or model of the individual's oral cavity where the dental prosthesis will fit; (b) fabricating a core dental prosthesis corresponding to the individual's scan, mold, or model of the oral cavity with a PMMA polymer; (c) generating microspheres comprised of PCL containing the compound or compounds necessary to treat or prevent the individual's oral or dental condition; (d) coating the PMMA core dental prosthesis with compound loaded PCL microspheres; (e) administering the completed dental prosthesis to the afflicted individual for sufficient time to treat the oral or dental condition; and (f) monitoring the individual's response to the dental prosthesis treatment.
Statement 17. A device, comprising: (a) a prosthesis; and (b) one or more microsphere(s) encapsulating one or more drug(s), where the one or more microsphere(s) are disposed in the prosthesis and/or on a surface of the prosthesis.
Statement 18. The device according to Statement 17, where the prosthesis is chosen from splints, mouth guards, full dentures, partial dentures, or fillings.
Statement 19. The device according to Statement 17 or Statement 18, where the one or more microsphere(s) comprise polycaprolactone (PCL) or polylactic acid. The microspheres of the present disclosure may be formed from chitosan, polyethylene, glass, polyphosphazene, or polymethacrylate, or be formed from liposomes.
Statement 20. The device according to Statement 19, where the one or more microsphere(s) is/are PCL. The microspheres of the present disclosure may be formed from PCL, polylactic acid, chitosan, polyethylene, glass, polyphosphazene, or polymethacrylate, or be formed from liposomes.
Statement 21. The device according to any one of Statements 17-20, where the one or more drug(s) are the same or different.
Statement 22. The device according to any one of Statements 17-21, where the one or more drug(s) are chosen from antimicrobial agent(s), antifungal agent(s), antibiotic(s), antiviral(s), analgesic(s), steroid(s), immune modulator(s), fluoride, and the like, and combinations thereof.
Statement 23. The device according to Statement 22, where the one or more drug(s) is an antifungal agent chosen from amphotericin B, flucytosine, fluconazole, ketoconazole, miconazole, terbinafine, griseofulvin, Clotrimazole, Econazole, Anidulafungin, Micafungin, Caspofungin, Butoconazole. Bifonazole, Clotrimazole, Econazole. Fenticonazole, Isoconazole, Ketoconazole, Luliconazole, Miconazole, Omoconazole, Oxiconazole, Sulconazole, Sertaconazole, Tioconazole, Albaconazole, Abafungin, amorolfin, butenafine, naftifine, terbinafine, Epoxiconazole, Efinaconazole, Fluconazole, Itraconazole, Isavuconazole, Propiconazole, Posaconazole, Ravuconazole, Terconazole, Voriconazole, and the like, and combinations thereof.
Statement 24. The device according to Statement 23 where the antifungal agent is amphotericin B.
Statement 25. The device according to any one of Statements 17-24, where the prosthesis comprises polymethylmethacrylate (PMMA).
Statement 26. The device according to any one of Statements 17-25, where the prosthesis is 3D printed.
Statement 27. A method of making a device comprising: (a) obtaining a scan, mold, or model of an individual's oral cavity where the dental prosthesis will fit; (b) fabricating a core dental prosthesis corresponding to the individual's scan, mold, or model of the oral cavity with PMMA polymer; (c) generating one or more microsphere(s)comprising one or more drug(s); and (d) coating the core dental prosthesis with the one or more microsphere(s)comprising one or more drug(s).
Statement 28. The method according to Statement 27, where the prosthesis is a denture.
Statement 29. The method according to Statement 27 or Statement 28, where the one or more drug(s) are chosen from antimicrobial agent(s), antifungal agent(s), antibiotic(s), antiviral(s), analgesic(s), steroid(s), immune modulator(s), fluoride, and the like, and combinations thereof.
Statement 30. The method according to Statement 29, where the one or more drug(s) is an antifungal agent chosen from amphotericin B, flucytosine, fluconazole, ketoconazole, miconazole, terbinafine, griseofulvin, Clotrimazole, Econazole, Anidulafungin, Micafungin, Caspofungin, Butoconazole. Bifonazole, Clotrimazole, Econazole. Fenticonazole, Isoconazole, Ketoconazole, Luliconazole, Miconazole, Omoconazole, Oxiconazole, Sulconazole, Sertaconazole, Tioconazole, Albaconazole, Abafungin, amorolfin, butenafine, naftifine, terbinafine, Epoxiconazole, Efinaconazole, Fluconazole, Itraconazole, Isavuconazole, Propiconazole, Posaconazole, Ravuconazole, Terconazole, Voriconazole, and the like, and combinations thereof.
Statement 31. The method according to Statement 30, where the antifungal agent is amphotericin B.
Statement 32. The method according to any one of Statements 27-31, where the one or more microsphere(s) comprise PCL. The microspheres of the present disclosure may be formed from PCL, polylactic acid, chitosan, polyethylene, glass, polyphosphazene, or polymethacrylate, or be formed from liposomes.
Statement 33. The method according to any one of Statements 27-32, where the fabricating includes 3D printing.
Statement 34. A method of treating an individual afflicted with or suspected of having an oral or dental condition comprising: (a) obtaining a scan, mold, or model of the individual's oral cavity where the dental prosthesis will fit; (b) fabricating a core dental prosthesis corresponding to the individual's scan, mold, or model of the oral cavity with a PMMA polymer; (c) generating one or more microsphere(s)comprising one or more drug(s); (d) coating the core dental prosthesis with the one or more microsphere(s)comprising one or more drug(s); (e) administering the completed dental prosthesis to the individual for sufficient time to treat the oral or dental condition; and (f) optionally, monitoring the individual's response to the dental prosthesis treatment.
Statement 35. The method according to Statement 34, where the individual is recovering from surgery and/or a dental procedure.
Statement 36. The method according to Statement 34 or Statement 35, where the individual has or is suspected of having a C. albicans infection.
Statement 37. The method according to any one of Statements 34-36, where the one or more drug(s) is an antifungal agent chosen from amphotericin B, flucytosine, fluconazole, ketoconazole, miconazole, terbinafine, griseofulvin, Clotrimazole, Econazole, Anidulafungin, Micafungin, Caspofungin, Butoconazole. Bifonazole, Clotrimazole, Econazole. Fenticonazole, Isoconazole, Ketoconazole, Luliconazole, Miconazole, Omoconazole, Oxiconazole, Sulconazole, Sertaconazole, Tioconazole, Albaconazole, Abafungin, amorolfin, butenafine, naftifine, terbinafine, Epoxiconazole, Efinaconazole, Fluconazole, Itraconazole, Isavuconazole, Propiconazole, Posaconazole, Ravuconazole, Terconazole, Voriconazole, and the like, and combinations thereof.
Statement 38. The method according to Statement 37, where the antifungal agent is amphotericin B.
Statement 39. The method according to any one of Statements 34-38, where the one or more microsphere(s) comprise PCL. The microspheres of the present disclosure may be formed from PCL, polylactic acid, chitosan, polyethylene, glass, polyphosphazene, or polymethacrylate, or be formed from liposomes.
Statement 40. A method of making a device comprising: (a) obtaining a scan, mold, or model of an individual's oral cavity where the dental prosthesis will fit; (b) fabricating a core dental prosthesis corresponding to the individual's scan, mold, or model of the oral cavity with PMMA polymer, where the PMMA polymer filament one or more microsphere(s) comprising one or more drug(s).
Statement 41. The method according to Statement 40, where the prosthesis is a denture.
Statement 42. The method according to Statement 40 or Statement 41, where the one or more drug(s) are chosen from antimicrobial agent(s), antifungal agent(s), antibiotic(s), antiviral(s), analgesic(s), steroid(s), immune modulator(s), fluoride, and the like, and combinations thereof.
Statement 43. The method according to Statement 42, where the one or more drug(s) is an antifungal agent chosen from amphotericin B, flucytosine, fluconazole, ketoconazole, miconazole, terbinafine, griseofulvin, Clotrimazole, Econazole, Anidulafungin, Micafungin, Caspofungin, Butoconazole. Bifonazole, Clotrimazole, Econazole. Fenticonazole, Isoconazole, Ketoconazole, Luliconazole, Miconazole, Omoconazole, Oxiconazole, Sulconazole, Sertaconazole, Tioconazole, Albaconazole, Abafungin, amorolfin, butenafine, naftifine, terbinafine, Epoxiconazole, Efinaconazole, Fluconazole, Itraconazole, Isavuconazole, Propiconazole, Posaconazole, Ravuconazole, Terconazole, Voriconazole, and the like, and combinations thereof.
Statement 44. The method according to Statement 43, where the antifungal agent is amphotericin B.
Statement 45. The method according to any one of Statements 40-44, where the one or more microsphere(s) comprise PCL. The microspheres of the present disclosure may be formed from PCL, polylactic acid, chitosan, polyethylene, glass, polyphosphazene, or polymethacrylate, or be formed from liposomes.
Statement 46. The method according to any one of Statements 40-45, where the fabricating includes 3D printing.
Statement 47. A method of treating an individual afflicted with or suspected of having an oral or dental condition comprising: (a) obtaining a scan, mold, or model of the individual's oral cavity where the dental prosthesis will fit; (b) fabricating a core dental prosthesis corresponding to the individual's scan, mold, or model of the oral cavity with a PMMA polymer; (c) administering the completed dental prosthesis to the individual for sufficient time to treat the oral or dental condition; and (d) optionally, monitoring the individual's response to the dental prosthesis treatment, where the PMMA polymer filament one or more microsphere(s) comprising one or more drug(s).
Statement 48. The method according to Statement 47, where the individual is recovering from surgery and/or a dental procedure.
Statement 49. The method according to Statement 47 or Statement 48, where the individual has or is suspected of having a C. albicans infection.
Statement 50. The method according to Statement 47-49, where the one or more drug(s) is an antifungal agent chosen from amphotericin B, flucytosine, fluconazole, ketoconazole, miconazole, terbinafine, griseofulvin, Clotrimazole, Econazole, Anidulafungin, Micafungin, Caspofungin, Butoconazole. Bifonazole, Clotrimazole, Econazole. Fenticonazole, Isoconazole, Ketoconazole, Luliconazole, Miconazole, Omoconazole, Oxiconazole, Sulconazole, Sertaconazole, Tioconazole, Albaconazole, Abafungin, amorolfin, butenafine, naftifine, terbinafine, Epoxiconazole, Efinaconazole, Fluconazole, Itraconazole, Isavuconazole, Propiconazole, Posaconazole, Ravuconazole, Terconazole, Voriconazole, and the like, and combinations thereof.
Statement 51. The method according to Statement 50, where the antifungal agent is amphotericin B.
Statement 52. The method according to any one of Statement 47-51, wherein the one or more microsphere(s) comprise PCL. The microspheres of the present disclosure may be formed from PCL, polylactic acid, chitosan, polyethylene, glass, polyphosphazene, or polymethacrylate, or be formed from liposomes.

The following description will provide specific examples of the present disclosure. Those skilled in the art will recognize that routine modifications to these embodiments can be made which are intended to be within the scope of the invention.

### EXAMPLE

The following provides examples of prosthetics of the present disclosure.

Materials and methods. Preparation of Amphotericin B-loaded PCL microspheres. PCL microspheres were prepared utilizing the double emulsion technique as described previously. Amphotericin B (125 μg/ml) and Gentamycin (5 mg/ml) solution (Thermo Fisher Scientific, NY, USA) solution was incorporated into Polycaprolactone (molecular weight 80,000; Sigma Aldrich, MO, USA) dissolved in dichloromethane (Sigma Aldrich, MO, USA).

Custom fabrication of PMMA filaments. Fast-curing polymethylmethacrylate tooth shade resin powder and liquid (LANGJET, IL, USA) were mixed in 2:1 ratio and drug-loaded PCL microspheres were added (0.2% w/v). The mixture was allowed to stand for 2 min to homogenize and polymerize that developed adequate viscosity to be loaded into a 10 ml syringe. The syringe was loaded onto a syringe pump and flow rate was set to 1.9 ml/min. The filament was passed through a bladeless fan to allow rapid cooling and maintaining extruded filament dimensions. These were allowed to further polymerize for 20 min and these filaments had diameters ranging from 2.85 to 3.00 mm.

3D printing using custom-fabricated PMMA filaments. A dual extrusion fused filament fabrication printer (BCN3D Sigma, Barcelona, Spain) was used for 3D printing. This printer has dual 0.4 mm printing nozzles, 65° C. printing bed temperature, and can print at 10 mm/s print speed. The nozzle temperature was set at 275° C. (PMMA). Various 3D printed formats were used to assess mechanical testing, drug release, and biomass assays. Routine STL (Stereolithography) files were generated using Autodesk Meshmixer (Autodesk Inc., CA, USA) and imported into Cura 3D slicing software (Ultimaker, Geldermalsen, Netherlands) and a GCode file was utilized for printing. A 3-unit denture model was downloaded as an STL file from GrabCAD.

Mechanical testing. To measure effects of 3D printing versus conventional processing of PMMA, flexural strength was tested using a universal testing machine (Instron 33R4204, Instron Corp, MA, USA). Samples were prepared in accordance with ISO 4049 standards (2 mm×2 mm×25 mm) with both 3D printing and routine processing. 3-point flexural test was performed with 8 replicates to assess statistical significance.

Functionalization of 3D printed surfaces. To demonstrate drug incorporation, fluorescent microspheres containing Protoporphyrin-IX (PpX, Sigma-Aldrich, MO, USA) were fabricated and custom PMMA filaments were synthesized as described in 2.3 above. These PMMA filaments were used to 3D print a partial denture and imaged with a fluorescence imaging system (ChemiDoc, Bio-Rad, CA, USA).

Drug incorporation and release. Presence of the drug within 3D printed PMMA was established with FTIR analyses (Spectrum 100, FT-IR Spectrometer, Perkin Elmer, MA, USA). For comparison, loaded and unloaded discs were utilized. 3D printed discs were placed in a 6-well plate and each well was filled with 5 ml of Dulbecco's Phosphate Buffered Saline (Corning VA, USA) with 0.2% of the sodium deoxycholate (Sigma Aldrich, MO, USA) at pH 7.4. Plates were sealed with parafilm and kept in the shaker at 37° C. at 50 rpm. Samples were withdrawn at regular intervals and media was replenished. Samples were analyzed by absorbance between 344 to 354 nm using a DU800 Spectrophotometer (Beckman Coulter, CA, USA). Drug quantitation was calculated by area under the curve analyses against a standard drug dilutions curve using NIH Image J software.

Scanning electron microscopy. Samples were sputter-coated with a thin carbon deposit under vacuum to provide conductivity, and then examined by using a field emission scanning electron microscope (SEM) (Hitachi S-4000), typically at 20 or 30 KeV.

Candida biofilm biomass assay. All samples were disinfected prior to studies with a 70% alcohol rinse and exposure to UV for 10 min. The disinfected discs were placed in a 12-well plate each with a 2.5 ml of the Yeast Nitrogen Base (YNB) media containing 2% glucose and 20% FBS (Seradigm, VWR, PA, USA) using 1×105 cells/ml of C. albicans (#SC5314, ATCC, VA, USA) and incubated for 48 h at 37° C. The media covering the biofilms growing on the well bottoms was removed and the biofilms themselves were removed using 1× PBS. They were then transferred to pre-weighed microfuge tubes. The samples were centrifuged and most of the liquid was removed to facilitate drying. The discs and the well bottom biofilm samples were placed in a desiccator jar with anhydrous Calcium Chloride (EMD Millipore Corporation, MA, USA) as the desiccant. Dry cell mass was determined after 3 days with a precision balance (Mettler-Toledo, OH, USA).

Statistical analyses. Data was collected in Excel (Microsoft, WA, USA) and analyzed using Student T-Test where p<0.05 was considered to be statistically significant.

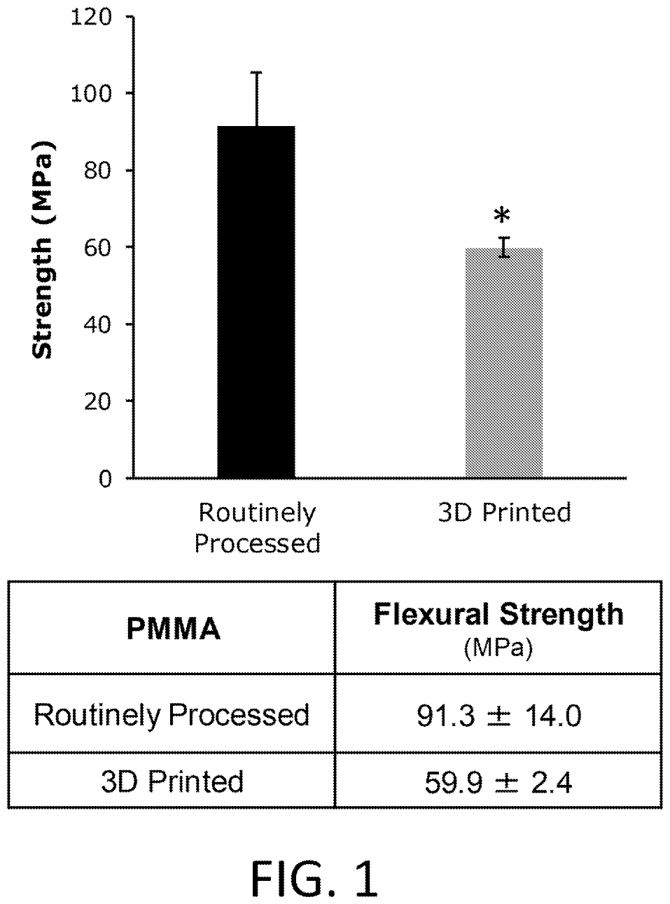

Results and discussion. Mechanical testing of custom fabricated PMMA filaments Flexural strength of PMMA by 3D printing was reduced by 35% (n=8, p<0.05) compared to conventional processing (FIG. 1). However, 3D printed samples did not reach a fracture point and would continue to flex inside the cell indicating increasing stress induced decreasing strain (plastic) on samples. This is specifically interesting as all routinely processed samples reached a fracture point during testing. The dual (chemical and thermoplastic) processing of PMMA during 3D printing most likely affects PMMA mechanical properties. One potential strategy to increase the PMMA properties would be to include fillers or cross-linkers that reinforce the polymer matrix. This clearly warrants further investigations to enable effective clinical translation of this specific 3D printing approach.

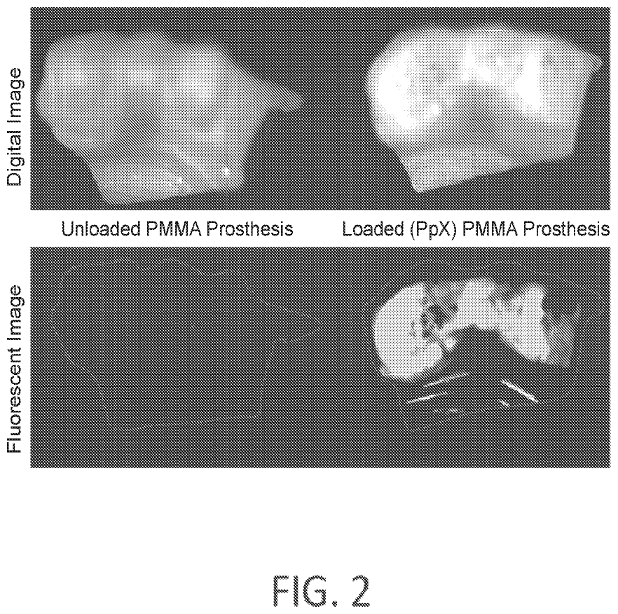

Functionalization of PMMA with PCL microspheres. The PpX-encapsulated PCL microspheres within PMMA filaments were used to print a partial (3 unit) denture and visualized using fluorescence imaging at 680 nm (FIG. 2). These data confirm the ability to utilize small molecules within microspheres that can be incorporated in PMMA to provide specific functionality. The encapsulation ensures protection and improved integrity of the payload during 3D-printing process. The wide range of biomolecular payloads can include drugs, peptides, nucleic acids, and other therapeutic substances. The distribution of the payload can also be specifically tailored to provide additional functionality of a given printed surface including combinatorial delivery of molecules from multiple surfaces may also be feasible.

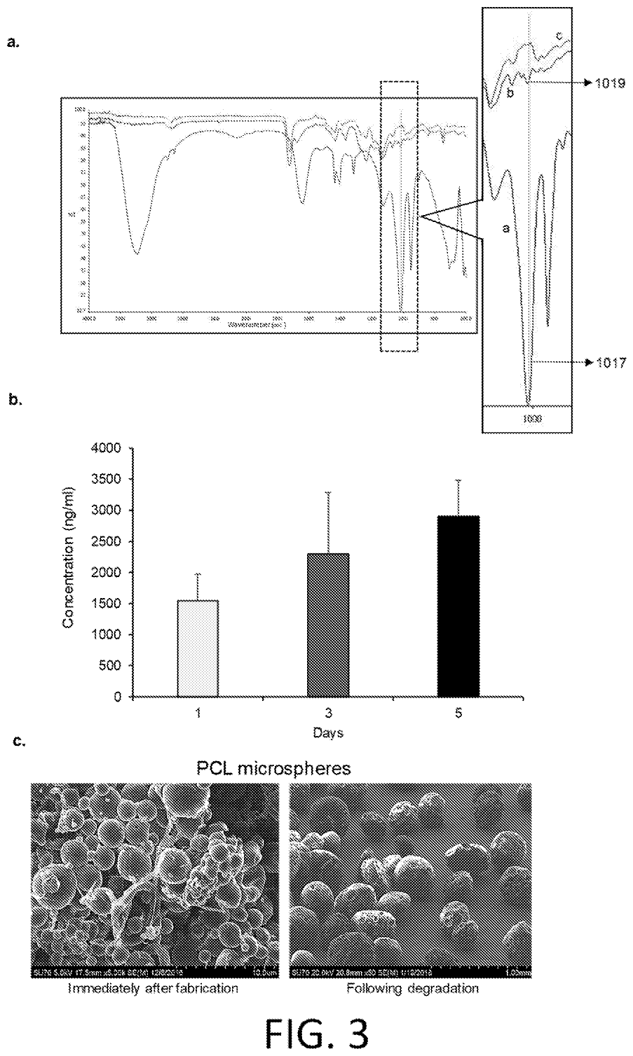

Presence of the drug in the printed disc and releasing medium. Loaded (AmB) and unloaded PMMA discs were analyzed by FTIR analyses and demonstrated the anticipated polymer spectra (FIG. 3a). Additionally, the AmB loaded discs showed peaks at 1017 cm⁻¹ that were evident in AmB (1019 cm⁻¹) correlating with the CeCeH and eCH bonds which were not present in the unloaded PMMA discs. It is interesting to note that the 5 and 10 layered samples did not demonstrate absorbance peak in the spectral region (325-425 nm). The drug released by 1 layer 3D printed sample was 1544.5±425.2, 2295.7±992.8 and 2901.7±575.6 ng/ml at day 1, 3 and 5 respectively (FIG. 3b). The PCL microspheres were noted to lose their integrity by surface degradation that contributes to the controlled release of AmB enabling sustained antifungal effects (FIG. 3c).

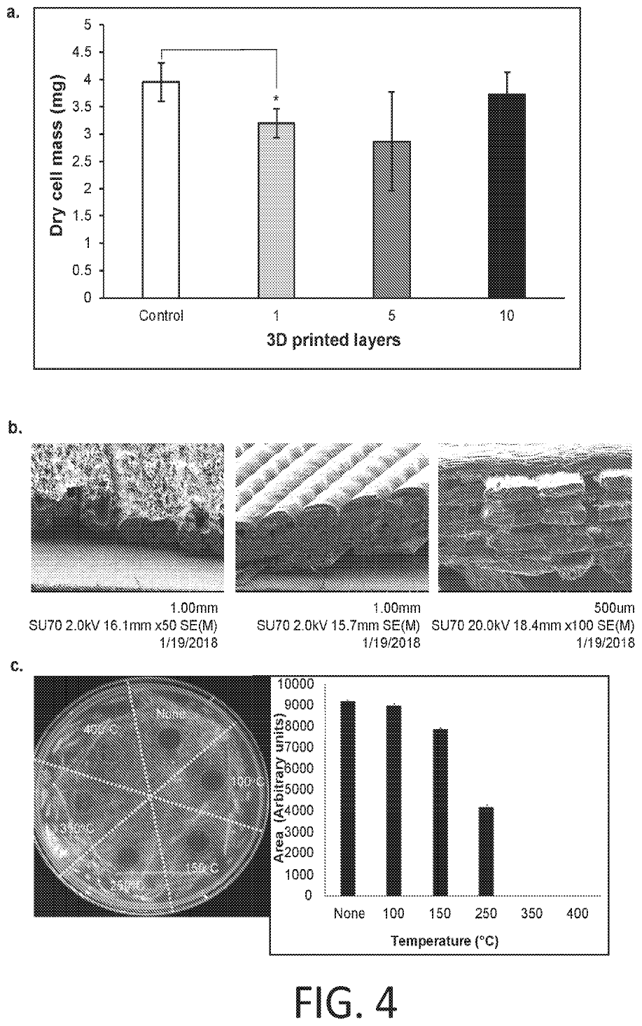

Biofilm biomass. The single layer AmB loaded disc had significantly (p<0.05) reduced biofilm biomass of C. albicans compared to the controls (FIG. 4a). Both 5 and 10 layered disc did not show any statistically significant reduction. These results were consistent with the drug release assays noted in 3.2 above. Scanning electron microscopy noted a more porous, permissive single layer while the multiple layers appear to form solid, impervious surfaces (FIG. 4b). Further, direct heat treatment of the AmB leads to reduced integrity of the drug as evidenced by reduced anti-fungal effects suggesting encapsulation and controlled release from PCL microspheres within the PMMA prosthesis will be useful (FIG. 4c). Together, these data demonstrate the feasibility of the PCL-PMMA additive 3D-printing technique with anti-fungal therapeutic properties.

Conclusions. There is strong interest in utilizing 3D printing, both additive and subtractive, in dentistry. The rapid progress in 3D printing technologies has enabled ease of operation, economical and affordable, shorter manufacturing times and digitization of complete prosthetics workflow. The ability to provide prostheses chair-side (in the clinic) is very attractive for both traditional and outreach healthcare settings. Functionalization of the 3D printed surfaces, anti-fungal in this case, provides previously unachievable resources for patient management. This specific anti-fungal application can be invaluable in highly susceptible populations such as immunocompromised or hospitalized patients, populations with compromised motor skills to maintain oral hygiene such as geriatric or disabled patients. Further, the ability to digitally capture and modify surfaces with 3D printing could enable modifications (relining) of the existing prosthesis as well. The relining can use 3D printing or can be a manual process. The potential prosthesis that can be 3D printed includes surgical splints (growth factors to promote healing), orthodontic appliances (fluoride leaching for anti-caries) or interim treatment prosthesis (with analgesics or corticosteroids).

Described herein, a modest drug release was achieved by controlling two parameters firstly, initial concentration within microspheres and rate of polymer (PCL) degradation and second, microsphere ratio (0.2% w/v) within PMMA filament. Printing multiple layers could additionally provide drug dosing. Reduced drug elution was observed from multilayer prosthesis by FTIR analyses, a solid, impervious surface layers with SEM and, most importantly, no significant anti-fungal activity in the biomass assay. While the precise reasons for this phenomenon needs further investigation, a potential contributing factor could be thermal characteristics of the PCL microspheres (tm 60° C.) and PMMA ((tm 85-165° C.)) polymer mix and potential persistence during multilayer printing. Nonetheless, additional approaches to stoichiometrically optimize drug delivery could involve changes to initial drug concentrations or ratio of PCL microspheres to PMMA as well as polymers with variable degradation rate such as Poly lactide-glycolic acid (PLGA). Other strategies to modify PMMA prostheses surfaces are being actively investigated.

The major impact of this innovative 3D additive printing system is its potential impact on saving cost and time for personnel and institutions. The rapid, customizable digital workflow can significantly reduce patient and clinic personnel time compared to conventional, protracted, multi-stage lab manufacturing. Further, this approach can be easily adapted to reline existing prostheses with additional tissue interface functionalities such as anti-microbial or promote optimal healing preventing the need for repeat prostheses. This suggests that the custom 3D printing approach will improve efficiency and sustainability in prostheses manufacturing. The exciting recent development of fungal detection systems suggests that future iterations of these anti-fungal prostheses could involve ‘sense’ and ‘respond’, smart systems for theranostics (therapy and diagnostics) enabling a precision-medicine approach. This is particularly relevant in the current era of antimicrobial resistance due to indiscriminate antibiotic use. This work noted successful incorporation of light-sensitive (PpX-incorporated) microspheres capable of temporal activation of these smart systems. In conclusion, the hybrid, additive 3D printed custom fabrication system described in this work can be a significant tool in the clinical armamentarium enabling broader, sustainable comprehensive applications to positively impact human health.

Although the present disclosure has been described with respect to one or more particular embodiments, it will be understood that other embodiments of the present disclosure may be made without departing from the scope of the present disclosure. Hence, the present disclosure is deemed limited only by the appended claims and the reasonable interpretation thereof.

What is claimed is:

1. A device, comprising:
(a) a prosthesis; and
(b) one or more microsphere(s) encapsulating one or more drug(s),
wherein the one or more microsphere(s) are disposed in the prosthesis and/or on a surface of the prosthesis.

2. The device of claim 1, wherein the prosthesis is chosen from a splint, mouth guard, full denture, partial denture, or filling.

3. The device of claim 1, wherein the one or more microsphere(s) comprise polycaprolactone (PCL), polylactic acid, chitosan, polyethylene, glass, polyphosphazene, or polymethacrylate.

4. The device of claim 1, wherein the one or more microsphere(s) is/are PCL.

5. The device of claim 1, wherein the one or more drug(s) are the same or different.

6. The device of claim 1, wherein the one or more drug(s) are chosen from antimicrobial agent(s), antifungal agent(s), antibiotic(s), antiviral(s), analgesic(s), steroid(s), immune modulator(s), fluoride, and combinations thereof.

7. The device of claim 6, wherein the one or more drug(s) is an antifungal agent chosen from amphotericin B, flucytosine, fluconazole, ketoconazole, miconazole, terbinafine, griseofulvin, Clotrimazole, Econazole, Anidulafungin, Micafungin, Caspofungin, Butoconazole. Bifonazole, Clotrimazole, Econazole. Fenticonazole, Isoconazole, Ketoconazole, Luliconazole, Miconazole, Omoconazole, Oxiconazole, Sulconazole, Sertaconazole, Tioconazole, Albaconazole, Abafungin, amorolfin, butenafine, naftifine, terbinafine, Epoxiconazole, Efinaconazole, Fluconazole, Itraconazole, Isavuconazole, Propiconazole, Posaconazole, Ravuconazole, Terconazole, Voriconazole, and the like, and combinations thereof.

8. The device of claim 7, wherein the antifungal agent is amphotericin B.

9. The device of claim 1, wherein the prosthesis comprises polymethylmethacrylate (PMMA).

10. The device of claim 1, wherein the prosthesis is 3D printed.

11. A method of making a device comprising:
(a) obtaining a scan, mold, or model of an individual's oral cavity where the dental prosthesis will fit;
(b) fabricating a core dental prosthesis corresponding to the individual's scan, mold, or model of the oral cavity with PMMA polymer;
(c) generating one or more microsphere(s)comprising one or more drug(s); and
(d) coating the core dental prosthesis with the one or more microsphere(s)comprising one or more drug(s).

12. The method of claim 11, wherein the prosthesis is a denture.

13. The method of claim 11, wherein the one or more drug(s) are chosen from antimicrobial agent(s), antifungal agent(s), antibiotic(s), antiviral(s), analgesic(s), steroid(s), immune modulator(s), fluoride, and combinations thereof.

14. The method of claim 13, wherein the one or more drug(s) is an antifungal agent chosen from amphotericin B, flucytosine, fluconazole, ketoconazole, miconazole, terbinafine, griseofulvin, Clotrimazole, Econazole, Anidulafungin, Micafungin, Caspofungin, Butoconazole. Bifonazole, Clotrimazole, Econazole. Fenticonazole, Isoconazole, Ketoconazole, Luliconazole, Miconazole, Omoconazole, Oxiconazole, Sulconazole, Sertaconazole, Tioconazole, Albaconazole, Abafungin, amorolfin, butenafine, naftifine, terbinafine, Epoxiconazole, Efinaconazole, Fluconazole, Itraconazole, Isavuconazole, Propiconazole, Posaconazole, Ravuconazole, Terconazole, Voriconazole, and the like, and combinations thereof.

15. The method of claim 14, wherein the antifungal agent is amphotericin B.

16. The method of claim 11, wherein the one or more microsphere(s) comprise PCL.

17. The method of claim 11, wherein the fabricating includes 3D printing.

18. A method of making a device comprising:
(a) obtaining a scan, mold, or model of an individual's oral cavity where the dental prosthesis will fit;
(b) fabricating a core dental prosthesis corresponding to the individual's scan, mold, or model of the oral cavity with PMMA polymer,
wherein the PMMA polymer filament one or more microsphere(s) comprising one or more drug(s).

19. The method of claim 18, wherein the prosthesis is a denture.

20. The method of claim 18, wherein the one or more drug(s) are chosen from antimicrobial agent(s), antifungal agent(s), antibiotic(s), antiviral(s), analgesic(s), steroid(s), immune modulator(s), fluoride, and combinations thereof.

21. The method of claim 20, wherein the one or more drug(s) is an antifungal agent chosen from amphotericin B, flucytosine, fluconazole, ketoconazole, miconazole, terbinafine, griseofulvin, Clotrimazole, Econazole, Anidulafungin, Micafungin, Caspofungin, Butoconazole. Bifonazole, Clotrimazole, Econazole. Fenticonazole, Isoconazole, Ketoconazole, Luliconazole, Miconazole, Omoconazole, Oxiconazole, Sulconazole, Sertaconazole, Tioconazole, Albaconazole, Abafungin, amorolfin, butenafine, naftifine, and terbinafine, Epoxiconazole, Efinaconazole, Fluconazole, Itraconazole, Isavuconazole, Propiconazole, Posaconazole, Ravuconazole, Terconazole, Voriconazole, and combinations thereof.

22. The method of claim 21, wherein the antifungal agent is amphotericin B.

23. The method of claim 18, wherein the one or more microsphere(s) comprise PCL.

24. The method of claim 18, wherein the fabricating includes 3D printing.

25. A method of treating an individual afflicted with or suspected of having an oral or dental condition comprising:
(a) obtaining a scan, mold, or model of the individual's oral cavity where the dental prosthesis will fit;
(b) fabricating a core dental prosthesis corresponding to the individual's scan, mold, or model of the oral cavity with a PMMA polymer;
(c) generating one or more microsphere(s)comprising one or more drug(s);
(d) coating the core dental prosthesis with the one or more microsphere(s)comprising one or more drug(s);
(e) administering the completed dental prosthesis to the individual for sufficient time to treat the oral or dental condition; and
(f) optionally, monitoring the individual's response to the dental prosthesis treatment.

26. The method of claim 25, wherein the individual is recovering from surgery and/or a dental procedure.

27. The method of claim 25, wherein the individual has or is suspected of having a C. albicans infection.

28. The method of claim 25, wherein the one or more drug(s) is an antifungal agent chosen from amphotericin B, flucytosine, fluconazole, ketoconazole, miconazole, terbinafine, griseofulvin, Clotrimazole, Econazole, Anidulafungin, Micafungin, Caspofungin, Butoconazole. Bifonazole, Clotrimazole, Econazole. Fenticonazole, Isoconazole, Ketoconazole, Luliconazole, Miconazole, Omoconazole, Oxiconazole, Sulconazole, Sertaconazole, Tioconazole, Albaconazole, Abafungin, amorolfin, butenafine, naftifine, and terbinafine, Epoxiconazole, Efinaconazole, Fluconazole, Itraconazole, Isavuconazole, Propiconazole, Posaconazole, Ravuconazole, Terconazole, Voriconazole, and combinations thereof.

29. The method of claim 28, wherein the antifungal agent is amphotericin B.

30. The method of claim 25, wherein the one or more microsphere(s) comprise PCL.

31. A method of treating an individual afflicted with or suspected of having an oral or dental condition comprising:
(a) obtaining a scan, mold, or model of the individual's oral cavity where the dental prosthesis will fit;
(b) fabricating a core dental prosthesis corresponding to the individual's scan, mold, or model of the oral cavity with a PMMA polymer;
(c) administering the completed dental prosthesis to the individual for sufficient time to treat the oral or dental condition; and
(d) optionally, monitoring the individual's response to the dental prosthesis treatment,
wherein the PMMA polymer filament one or more microsphere(s) comprising one or more drug(s).

32. The method of claim 31, wherein the individual is recovering from surgery and/or a dental procedure.

33. The method of claim 31, wherein the individual has or is suspected of having a C. albicans infection.

34. The method of claim 31, wherein the one or more drug(s) is an antifungal agent chosen from amphotericin B, flucytosine, fluconazole, ketoconazole, miconazole, terbinafine, griseofulvin, Clotrimazole, Econazole, Anidulafungin, Micafungin, Caspofungin, Butoconazole. Bifonazole, Clotrimazole, Econazole. Fenticonazole, Isoconazole, Ketoconazole, Luliconazole, Miconazole, Omoconazole, Oxiconazole, Sulconazole, Sertaconazole, Tioconazole, Albaconazole, Abafungin, amorolfin, butenafine, naftifine, and terbinafine, Epoxiconazole, Efinaconazole, Fluconazole, Itraconazole, Isavuconazole, Propiconazole, Posaconazole, Ravuconazole, Terconazole, Voriconazole, and combinations thereof.

35. The method of claim 34, wherein the antifungal agent is amphotericin B.

36. The method of claim 31, wherein the one or more microsphere(s) comprise PCL.
